(19) United States
(12) Patent Application Publication
(10) Pub. No.: US 2011/0081386 A1
(43) Pub. Date: Apr. 7, 2011

(54) CONTROLLED RELEASE OF ACTIVE AGENTS FROM OLEOSOMES

(75) Inventors: Jacob Guth, Upper Black Eddy, PA (US); John Ferguson, Lawrenceville, PA (US)

(73) Assignee: SEMBIOSYS GENETICS, INC.

(21) Appl. No.: 12/935,069

(22) PCT Filed: Apr. 10, 2009
(86) PCT No.: PCT/US09/02242

§ 371 Date: Dec. 2, 2010

Related U.S. Application Data
(60) Provisional application No. 61/071,109, filed on Apr. 11, 2008.

(51) Int. Cl. A61K 8/11; A61K 9/00; A61Q 19/00; A61P 17/00; A23D 9/04; A23P 1/04

(52) U.S. Cl. 424/400; 426/98; 426/302

(57) ABSTRACT

The release rate of an active agent from oleosomes can be modulated by formulation of the oleosomes with a release control agent, such as a multihydric alcohol. In this context, oleosomes containing an active agent may be used in the preparation of a variety of formulations, including formulations for topical use.

### FIELD OF THE INVENTION

The present invention relates to novel compositions, comprised of oleosomes, and to methodology for their manufacture. The inventive compositions are useful for, inter alia, the manufacture of products for topical application to surface area of the human body.

### BACKGROUND OF THE INVENTION

Plant seed oils, such as palm oil, sunflower oil and rapeseed (Canola) oil, are a major agricultural commodity worldwide with a large variety of industrial and nutritional uses. More than 15 billion pounds of plant seed oil are produced annually in the United States alone. Wallis J., et al., “Seed oils and their metabolic engineering,” in: SEED TECHNOLOGY AND ITS BIOLOGICAL BASIS, M. Black & J. D. Bewley (eds.), Sheffield Biological Sciences (2000). Ninety eight percent of the plant seed oil production in the United States is used is for nutritional purposes, such as in the manufacture of cooking oil and margarine. The balance of plant oils are used as raw materials in the manufacture of industrial products such as soaps, plasticizers, polymers, surfactants and lubricants.

Plant oils are triacylglycerols, i.e., a glycerol moiety in which each of the hydroxyl groups is esterified to a fatty acid. The glycerol backbone of the triacyl glycerol is invariable in structure, but the fatty acids attached to the glycerol varies considerably depending on the plant oil.

The structure of the fatty acid determines the physical and chemical properties of the plant oil. For example, the number of double bonds in a fatty acid, a variable frequently referred to as the “degree of unsaturation,” affects the melting point of oils, while the chain length of a fatty acid affects influences its viscosity, lubricity and solubility.

Triacylglycerol molecules are insoluble in aqueous environments and tend to coalesce into oil droplets. In order to store these water insoluble triacylglycerols, plants have developed unique seed oil storage compartments of approximately 1-10 μm in diameter within the plant seed cells, variously known as “oil bodies,” “oleosomes,” “lipid bodies,” and “spherosomes” (collectively, “oleosomes”). See Huang, Ann. Rev. Plant Mol. Biol. 43: 177-200 (1992). In addition to plant oil, these oleosomes comprise two chemical constituents: phospholipids and a class of proteins, known to the art as oil body proteins. From a structural standpoint, oleosomes are a triacylglycerol core encapsulated by a half unit membrane of phospholipids, in which the oil body proteins are embedded. Oil body proteins are believed to play a role in preventing the oleosomes from coalescing into much larger oil droplets.

For extraction of plant oils, the seeds are crushed or pressed and then refined, using processes typically involving the use of organic solvents to separate the plant oil from other seed constituents, such as seed proteins and carbohydrates. Non-organic solvent-based plant oil extraction methodologies also have been developed, as described, for example, by Embong and Jelen, Can. Inst. Food Sci. Techn. J. 10: 239-43 (1977).

Since the primary objective of these extraction processes is to obtain pure plant oil, however, they typically disrupt oleosome structure. Thus, conventional compositions prepared from plant oils generally do not comprise intact oleosomes.

For instance, U.S. Pat. No. 5,683,740 and No. 5,613,583 to Voultoury et al. disclose emulsions prepared from crushed seeds of oleaginous plants comprising lipidic vesicles. In the course of the crushing process described in these patents, the oleosomes substantially lose their structural integrity. Accordingly, it is disclosed that in the crushing process 70% to 90% of the seed oil is released in the form of free oil.

On the other hand, oleosomes that are isolated from plant seeds in a structurally intact form have a recognized, practical utility. Notably, oleosomes permit the formulation of complex mixtures of aqueous compounds and oil, in the absence of exogenous emulsifiers, at room temperature, see PCT Application 2005/097059 to Guth et al., and oleosomes may be loaded with active ingredients, as described by Murray et al., PCT Application 2005/030169.

A non-destructive methodology for preparing oleosomes is disclosed by Deckers et al. in U.S. Pat. No. 6,146,645, No. 6,183,762, No. 6,210,742, No. 6,372,234, No. 6,582,710, No. 6,596,287, No. 6,599,513 and No. 6,761,914 (collectively, the “Deckers Patents”). In accordance with the Deckers Patents, a purified oleosome preparation may be obtained and used to prepare emulsions in the presence of a multiplicity of other substances, in order to achieve a desirable balance of emulsification, viscosity and appearance and render these emulsions suitable for cosmetic, food, pharmaceutical, and industrial applications, inter alia.

It frequently is desirable to prepare oleosomes that contain active ingredients. Such preparations may be obtained by mixing the active ingredient with the oleosomes or by using optimized methods that result in the selective partitioning of the active ingredient into the oleosomes, as described in PCT Application 2005/030169. Encapsulation of active ingredients in oleosomes is advantageous for various reasons. For example, it permits the stabilization of traditionally unstable active ingredients, as well as the separation of agents that are harmful upon contact with each other.

Thus obtained, an oleosome preparation that comprises an active ingredient can be used as an ingredient to prepare finished formulations. Use of such a finished formulation, for example, in its application to the skin or its ingestion, will result in disruption of oleosome structure and the resultant release of the active ingredient.

In such an instance, it can be desirable to control the release of the active agent(s) from the oleosomes. Yet, in a given composition the oleosomes offer only the release profile that is inherent to the oleosome preparation, which typically entails a relatively rapid release of the active upon delivery of the oleosome.

In pharmaceutical and other contexts, such a rapid release, followed by a sharply declining release rate of the active, represents a suboptimal delivery of the active. Instead, a more gradual and sustained release of the active ingredient often is desirable.

Conventional oleosome preparation can not achieve selective control of the release of the active ingredient from constituent oleosome. In other words, it has been infeasible to effect a variable adaptation of the release kinetics of active ingredient from the oleosome, such that differing active ingredient properties and differences in the onset and duration of action of an active ingredient can be taken into consideration, to achieve optimal release kinetics.

These shortcomings in available methodology engender a need for another approach to preparing active-containing oleosomes compositions. In particular, it has been unclear how or even whether an oleosome preparation might be obtained that afforded the controlled release of a constituent active agent.

### SUMMARY OF THE INVENTION

The present invention provides novel formulations comprising oleosomes. The inventive formulations offer a system that allows for the controlled release of active agents from oleosomes.

Accordingly, the present invention encompasses a method for the release of an active agent from an oleosome, comprising:
(i) providing an oleosome preparation comprising an encapsulated active agent and a release control agent; and
(ii) applying the oleosome formulation to a surface such that the active agent is released from the oleosome formulation.

In preferred embodiments, the average rate of release of the active agent from the oleosomes is at least 15% less in the presence of the release control agent, when compared to the average rate of release of the active agent in the absence of the release control agent. The average rate of release can be measured by hexane extraction, for instance, as further discussed below.

In further preferred embodiments, the release control agent is a multihydric alcohol. Preferably, the multihydric alcohol is a non-aromatic diol, a non-aromatic triol or a non-aromatic polyol or a non-halogenated multi-hydric alcohol. In particularly preferred embodiments, the control release agent is glycerin or PEG.

Active agents that may be used in the invention vary and may be as desired. In general, a “active agent” in this context, when delivered to a living organism, exerts a detectable biological effect, including but not limited to a pharmacological effect.

The release control kinetics of the active agent may be optimized to obtain an oleosome preparation with particularly desired release kinetics by determining the release kinetics of the active at various concentrations of release control agent dispersed within a plurality of oleosome preparations. Accordingly, the present invention also contemplates a methodology for obtaining an oleosome preparation, comprised of an active agent, where the release kinetics of the active agent have been optimized. The inventive methodology comprises:
(i) preparing a plurality of oleosome preparations, each comprising an active agent and a release control agent, where the concentration of release control agent varies from preparation to preparation in the plurality;
(ii) (ii) determining the release kinetics of the active agent in each oleosome preparation; and
(iii) (iii) selecting from the plurality of oleosome preparations an oleosome preparation with optimized active agent release kinetics.

In another aspect, the present invention further provides novel compositions comprising (i) oleosomes; (ii) an active agent; and (iii) a release control agent.

The oleosome preparations obtained in accordance with the present invention are useful in the manufacture of a multiplicity of products, such as cosmetic products, food products, agriculture products, household products, inks, coatings, paints, pharmaceutical products and industrial products, inter alia.

Other features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those of skill in the art from this detailed description.

### BRIEF DESCRIPTION OF THE DRAWINGS

FIG. 1 depicts a particle size analysis of drying oleosomes in the presence and absence of glycerin, respectively.

FIG. 2 depicts particle size analysis of OMC-loaded oleosomes mixed with 15% distilled water, PEG 200, or glycerin. Samples were analyzed after release (2 hours at 35° C.).

FIG. 3 depicts particle size analysis of OMC-loaded oleosomes mixed with 5%, 10%, 15% or 20% PEG 200. Samples were analyzed after release (2 hours at 35° C.). The 2008-094 sample was the unloaded oleosomes, and the 15% OMC is the starting stock of loaded oleosomes before the addition of release control agents. Both controls were not exposed to release conditions.

FIG. 4 depicts particle size analysis of OMC-loaded oleosomes that were mixed with 10% glycerin or PEG 200 and then incubated at 35° C. for up to 4 hours. “Time 0” represents the samples prior to drying.

### DETAILED DESCRIPTION OF THE INVENTION

As mentioned above, this invention relates to methodology for the controlled release of active ingredients from formulations comprised of oleosomes. The present inventors have found that, for a preparation comprised of oleosomes that encapsulate an active ingredient, the release of the active ingredient from the oleosomes can be controlled by the inclusion of an agent, such as a multihydric alcohol, that affects the rate of water evaporation from the oleosome preparation (“release control agent”), such that faster evaporation correlates with faster release, and vice versa. In other words, an agent that brings about faster evaporation has been discovered to cause oleosomes, as the preparation dries, to lose integrity and release encapsulated active ingredient more rapidly. Thus, an oleosome preparation of the invention allows for control over the kinetics of release of an active ingredient from the oleosomes, which in turn permits optimization of the duration and action of the active ingredient.

Accordingly, the present invention provides a method for the release of an active agent from an oleosome. The inventive method comprises:
(i) providing an oleosome preparation comprising an encapsulated active agent and a release control agent; and
(ii) applying the oleosome formulation to a surface whereby the active agent is released from the oleosome formulation.

The oleosome preparation obtained in accordance with the invention is characterized by a controlled release of the active agent. Preferably the average rate of release of the active agent from the oleosomes is at least 15% less in the presence of the release control agent, compared to such average rate of release in the absence of the release control agent.

### Oleosome Preparation

The term “oleosome” here denotes any discrete, subcellular oil or wax storage organelle obtainable from a living cell. For present purposes, oleosomes may be obtained from any cell containing such organelles, including plant cells, fungal cells, yeast cells (Leber, R. et al., 1994, Yeast 10: 1421-28), bacterial cells (Pieper-Fürst et al., 1994, J. Bacteriol. 176: 4328-37), and algae cells (Roessler, P. G., 1988, J. Phycol. (London) 24: 394-400).

In preferred embodiments of the invention, oleosomes are obtained from a plant cell, where “cell” is inclusive of cells from pollen, spores, seed and vegetative plant organs, respectively, in which oleosomes are present. Generally, see Huang, Ann. Rev. Plant Physiol. 43: 177-200 (1992). More preferably, the oleosomes employed in the invention are obtained from a plant seed.

Among the plant seeds useful herein preferred are those seeds obtainable from plant species selected from the group of plant species consisting of almond (Prunus dulcis); anise (Pimpinella anisum); avocado (Persea spp.); beach nut (Fagus sylvatica); borage (Boragio officinalis); Brazil nut (Bertholletia excelsa); candle nut (Aleuritis tiglium); carapa (Carapa guineensis); cashew nut (Ancardium occidentale); castor (Ricinus communis); coconut (Cocus nucifera); coriander (Coriandrum sativum); cottonseed (Gossypium spp.); crambe (Crambe abyssinica); Crepis alpina; croton (Croton tiglium); cucumber (Cucumis sativus); Cuphea spp.; dill (Anethum gravealis); Euphorbia lagascae; evening primrose (Oenothera biennis); Dimorphoteca pluvialis; false flax (Camolina sativa); fennel (Foeniculum vulgaris); groundnut (Arachis hypogaea); hazelnut (coryllus avellana); hemp (Cannabis sativa); honesty plant (Lunnaria annua); jojoba (Simmondsia chinensis); kapok fruit (Ceiba pentandra); kukui nut (Aleuritis moluccana); Lesquerella spp., linseed/flax (Linum usitatissimum); lupin (Lupinus spp.); macademia nut (Macademia spp.); maize (Zea mays); meadow foam (Limnanthes alba); mustard (Brassica spp. and Sinapis alba); olive (Olea spp.); oil palm (Elaeis guineeis); oiticia (Licania rigida); paw paw (Assimina triloba); pecan (Juglandaceae spp.); perilla (Perilla frutescens); physic nut (Gatropha curcas); pilinut (Canarium ovatum); pine nut (pine spp.); pistachio (Pistachia vera); pongam (Bongamin glabra); poppy seed (Papaver soniferum); pumpkin (Cucurbita pepo); rapeseed (Brassica spp.); safflower (Carthamus tinctorius); sesame seed (Sesamum indicum); soybean (Glycine max); squash (Cucurbita maxima); sal tree (Shorea rubusha); Stokes aster (Stokesia laevis); sunflower (Helianthus annuus); tukuma (Astocarya spp.); Lung nut (Aleuritis cordata); vernonia (Vernonia galamensis); and mixtures thereof. Most preferably the plant seeds are from the group of plant species comprising: rapeseed (Brassica spp.), soybean (Glycine max), sunflower (Helianthus annuus), oil palm (Elaeis guineeis), cottonseed (Gossypium spp.), groundnut (Arachis hypogaea), coconut (Cocus nucifera), castor (Ricinus communis), safflower (Carthamus tinctorius), mustard (Brassica spp. and Sinapis alba), coriander (Coriandrum sativum), squash (Cucurbita maxima), linseed/flax (Linum usitatissimum), Brazil nut (Bertholletia excelsa), jojoba (Simmondsia chinensis), maize (Zea mays), crambe (Crambe abyssinica) and eruca (Eruca sativa). Most preferred in this context are oil bodies prepared from safflower (Carthamus tinctorius).

In order to prepare oleosomes from plants, the latter are grown and allowed to set seed, pursuant to conventional agricultural cultivation practices. After harvesting the seed and, if desired, removing material such as stones or seed hulls (dehulling), for example, by sieving or rinsing, and optionally drying of the seed, the seeds subsequently are processed by mechanical grinding. Preferably, a liquid phase is added prior to grinding of the seeds. This is known as “wet milling.” Wet milling in oil extraction processes has been reported for seeds from a variety of plant species including mustard (Aguilar et al. 1991, J. Texture Studies 22: 59-84), soybean (U.S. Pat. No. 3,971,856, Cater et al., 1974, J. Am. Oil Chem. Soc. 51: 137-41), peanut (U.S. Pat. No. 4,025,658 and No. 4,362,759), cottonseed (Lawhon et al., 1977, J. Am. Oil Chem. Soc. 54: 75-80), coconut (Kumar et al., 1995, INFORM 6: 1217-40), and safflower (U.S. Pat. No. 6,146,645).

Preferably, the liquid is water, although organic solvents such as ethanol may also be used. It also may be advantageous to imbibe the seeds for a time period from about fifteen minutes to about two days in a liquid phase prior to grinding. Imbibing may soften the cell walls and facilitate the grinding process. Imbibition for longer time periods may mimic the germination process and result in certain advantageous alterations in the composition of the seed constituents.

The seeds are preferably ground using a colloid mill. Besides colloid mills, other milling and grinding equipment capable of processing industrial scale quantities of seed may also be employed in the here described invention including: disk mills, colloid mills, pin mills, orbital mills, IKA mills and industrial scale homogenizers. The selection of the mill may depend on the seed throughput requirements as well as on the source of the seed that is employed.

Pursuant to the invention, it is important that seed oil bodies remain intact during the grinding process. Any operating condition, commonly employed in oil seed processing, that tends to disrupt oil bodies is unsuitable for a process of the invention, therefore.

Milling temperatures are preferably between 10° C. and 90° C. More preferably, they are between 15° C. and 50° C. and most preferably between 18° C. and 30° C., while the pH is preferably maintained between 2.0 and 11.0, more preferably between 6.0 and 9.0, and most preferably between 7.0 and 9.0.

Solid contaminants, such as seed hulls, fibrous material, undissolved carbohydrates and proteins, and other insoluble contaminants are removed from the ground seed fraction. Separation of solid contaminants may be accomplished using a decantation centrifuge. Depending on seed throughput requirements, the capacity of the decantation centrifuge may be varied by using other models of decantation centrifuges, such as 3-phase decanters. Operating conditions vary depending on the particular centrifuge which is employed and must be adjusted so that insoluble contaminating materials sediment and remain sedimented upon decantation. A partial separation of the oil body phase and liquid phase may be observed under these conditions.

After the removal of insoluble contaminants, the oleosome fraction is separated from the aqueous phase. In one embodiment of the invention, a tubular bowl centrifuge is employed. In a preferred embodiment, a disc stack centrifuge is employed. Alternatively, hydrocyclones or a settling of phases under natural gravitation or any other gravity-based separation technique. It also is possible to separate the oleosome fraction from the aqueous fraction via a size-exclusion method such as filtration, e.g., membrane ultrafiltration and crossflow microfiltration.

When a centrifuge is used for this purpose, an important parameter is the size of the ring dam employed used to operate the centrifuge. Ring dams are removable rings with a central, circular opening of varying size, and they regulate the separation of the aqueous phase from the oleosome phase, thereby governing the purity of the oleosome fraction that is obtained. The chosen ring dam size depends on the type of centrifuge and the type of oil seed used, as well as on the desired final consistency of the oleosome preparation.

In accordance with one embodiment of the invention, safflower oleosomes are obtained using an SA-7 (Westfalia) disc stack centrifuge in conjunction with a ring dam sizes of 69-75 mm. The efficiency of separation is further affected by the flow rate, which, in this embodiment, typically is maintained between 0.5 to 7.0 l/min. Temperatures are preferably maintained between 26° C. and 40° C.

Depending on the model centrifuge used, flow rates and ring dam sizes can be adjusted so that an optimal separation is achieved of the oleosome fraction from the aqueous phase. These adjustments will be readily apparent to a person knowledgeable in the field of process engineering.

Separation of solids and separation of the aqueous phase from the oleosome fraction may be carried out concomitantly. This can be done by means of a gravity-based separation method such as 3-phase tubular bowl centrifuge, a decanter, a hydrocyclone, or a size exclusion-based separation method.

An oleosome composition obtained at this stage in the inventive process generally is relatively crude, comprising numerous seed proteins, glycosylated and non-glycosylated, and other contaminants such as glucosinilates or its breakdown products. The invention comprehends such a composition but, in preferred embodiments, a substantial amount of seed contaminants is removed before preparing a stabilized oleosome preparation.

To accomplish removal of contaminating seed material, an oil oleosome preparation obtained upon separation from the aqueous phase, as described above, is washed at least once by resuspending the oleosome fraction in a liquid phase and centrifuging the resuspended fraction, which yields a “washed oleosome preparation.”

Pursuant to the invention, washing conditions are selected generally as a function of the desired purity of the oleosome preparation. In this regard, conditions that may be varied in a controlled manner, thereby to obtain oleosome preparations of differing degrees of oleosome purity, include the makeup of the liquid phase used for washing, the washing time, the ratio of liquid phase to oleosome phase, and pH. For example, the liquid phase may be water or an organic solvent. Typically, it is advantageous to select a buffered liquid phase that (i) has a pH removed at least one pH point from the isoelectric point of the oleosomes, which point generally varies between 4 and 6, depending on the oleosome source, because such conditions facilitate separation between oleosomes and contaminants. It also is advantageous that a buffered liquid phase (ii) have a pH at which oleosomes are stable, i.e., generally in the slightly basic pH range (pH 7.0-9.0).

Suitable buffer systems for this invention are illustrated by systems comprised of sodium chloride in concentrations between 0.01 M and at 2 M, sodium bicarbonate buffers at a concentration between 25 mM and 50 mM; and low salt buffers such as 50 mM Tris-HCl at pH 7.5. In an instance when safflower oleosomes are prepared, a 45 mM sodium bicarbonate buffer at pH 8.2 is particularly suitable for obtaining relatively pure oleosome preparations.

With such a buffer one can obtain, for instance, an oleosome preparation comprising 2% or less of non-oil body proteins. Additional conditions that influence oleosome purity, in accordance with this invention, are washing time and the relative quantities of oleosome/liquid phase. By extending the washing times and/or increasing the number of washes, and by using large amounts of liquid phase, it typically is possible to obtain a higher degree of oleosome purity, albeit at the expense of yield, as one skilled in process engineering would appreciate.

Washing conditions may be adjusted as a function of the source for the prepared oleosomes. In particular, the above-described parameters of buffer composition, washing time, pH and the like may be varied to influence the constitution of the oleosome preparation, as well as the contaminating constituents, since these vary as a function of the source.

Thus, as a function of the washing conditions, an “essentially pure” oleosome preparation can be obtained; that is, the only proteins present are oil body proteins. In a preferred embodiment, the oleosome fraction contains less than 30% (w/w) of non-oil body proteins, more preferably less than 20% (w/w), and even more preferably less than 10% (w/w). In a most preferred embodiment, the oleosome fraction comprises 2% (w/w) or less of non-oilbody proteins.

Washing at a number of different pH values may be beneficial, since this will allow the step-wise removal of contaminants, particularly proteins. SDS gel electrophoresis or other analytical techniques may conveniently be used to monitor the removal of seed proteins and other contaminants upon washing of the oleosomes. Also, in instances where more than one washing step is carried out, washing conditions may vary for different washing steps.

It is not necessary to remove all of the aqueous phase between washing steps and the final washed oleosome preparation may be suspended in water, a buffer system, for example, 50 mM Tris-HCI pH 7.5, or any other liquid phase. If so desired the pH may be adjusted to any pH between pH 2.0 and 11.0, preferably between 6.0 and 9.0, and most preferably between 7.0 and 8.5. The amount of water in the oleosome preparation may be varied and, depending on the amount of water, a more or less viscous oleosome preparation can be obtained, in accordance with the invention. Thus, oleosome preparations of the invention preferably contain more than 10% and less than 65% water by volume, more preferably more than 15% and less than 50% water by volume, and most preferably more than 20% water by volume and less then 50% water by volume.

Pursuant to the invention, the process for manufacturing an oleosome preparation may be performed in batch operations or in a continuous flow process. Particularly when a disc stack centrifuge is used, a system of pumps is conveniently set up to generate a continuous flow. Illustrative of the pumps that can be employed are an air-operated, double-diaphragm pump and a hydraulic, positive-displacement or peristaltic pump.

In order to maintain a supply of homogenous consistency to the decantation centrifuge and to the tubular bowl centrifuge, homogenizers such as an IKA homogenizer may be added between the separation steps. In-line homogenizers also may be added in between various centrifuges or size exclusion-based separation devices that are employed to wash the oil body preparations. Ring dam sizes, buffer compositions, temperature and pH may differ in each washing step.

An oleosome preparations obtained in accordance with the foregoing may be used via the approach described in greater detail below.

### Release Control Agents

As discussed above, a control release agent impacts on such release kinetics by influencing the rate of water evaporation from the inventive composition. In this context, the phrase “release control agent” denotes a substance that, when mixed with a composition of oleosomes that contain or “encapsulate” an active agent, modulates the release kinetics of the active agent from the oleosomes, relative to a composition that lacks the control release agent. Illustrative in this regard are ingredients that alter the boiling point (vapor pressure) of water and, hence, affect the rate of water evaporation when employed in this invention. A control release agent thus may reduce the release rate or increase it. For example, methyl, ethyl, and isopropyl alcohol increase vapor pressure, thereby speeding release, whereas glycerin, ethylene glycol and propylene glycol decrease vapor pressure, retarding release. In general terms, therefore, different release control agents can exhibit different release kinetics by virtue of affecting the water evaporation rate differently.

In a preferred embodiment, the control release agent is a multihydric alcohol. A “multihydric alcohol” is a hydroxyl-containing organic compound with two or more hydroxyl groups. While any suitable multihydric alcohol can be used, it is preferably a non-halogenated multihydric alcohol, and preferably of small-to-medium molecular weight, i.e., less than 50,000 Daltons. Thus, the multihydric alcohol is suitably a non-aromatic diol, triol, or polyol.

When the multihydric alcohol is a diol, it may be glycol or a non-aromatic glycol derivative. Suitable glycol derivatives are butylene glycol, polyethylene glycol, propylene glycol, hexylene glycol, dipropylene glycol, hexanediol, or polybutylene glycol.

When it is a triol, the multihydric alcohol can be 1, 2, 6 hexanetriol or glycerol. Polymers of glycerol also may be used, e.g., di, tri, tetra, penta, hexa, septa, octa, nona, or decaglycerol, as may be a lightly substituted derivative of glycerol and polymers thereof.

When the multihydric alcohol is a polyol, preferably at least one carbon atom does not have a hydroxyl group bound thereto. Exemplary of polyols in which a hydroxyl group is bound to every carbon atom are glycerol and sugars such as sorbitol. Some ethoxylates of such polyols are suitable for use in the formulations of the present invention, provided they are liquid at room temperature are or water soluble, for instance, sorbeth 6, sorbeth 20, sorbeth 30, and sorbeth 40. Examples of other polyols that may be used in accordance herewith include polyvinyl alcohols.

It is feasible to use multihydric sugars in the invention, including monosaccharidic sugars, such as glucose and fructose, and disaccharidic sugars such as sucrose, as well as complex multihydric sugars such as starch and cellulose. In addition, lightly substituted sugar esters may be used, provided that such esters remain multihydric.

The multihydric alcohol employed in the invention preferably is chosen from glycerol and its linear and non-linear polymeric analogues. A person knowledgeable in process engineering can identify multihydric alcohols, including others than those specifically mentioned here, as well as mixtures of multihydric alcohols, that also may be used in the invention without departing from its spirit and scope, pursuant to the present disclosure.

### Active Agents

In principle, any exogenous active ingredient may be used in accordance with the invention. “Active,” “active agent,” and “active ingredient” as used here to indicate any compound that, when delivered to a surface area, has a detectable physical or chemical effect with respect to the surface area, including any biological, physiological, pharmacological, therapeutic, or prophylactic effect. Thus, the active may be capable of enhancing or improving the physical appearance, health, fitness or performance characteristics of any surface area.

In certain embodiments the surface area is the interior or exterior of the human body or other mammal, e.g., human skin, hair, scalp, teeth and nails.

Any active that can be encapsulated in oleosomes may be used. In this regard, “encapsulated” means that the active is located, in whole or in part, within the triacyl glyceride core of the oleosome or within the lipid membrane of the oleosome.

In a preferred embodiment, the active is a hydrophobic compound, i.e., a compound which is not readily dissolved in polar solvents such as water. A common measure used to quantify the relative hydrophobicity of a compound is the LogP value, which reflects the ratio of the relative concentration of a compound in octanol and water when such compound is dissolved in a two phase water/octanol system. In preferred embodiments the LogP value of the active used in accordance herewith ranges from 0 to 8; in a more preferred embodiment the LogP value of the active ranges from 1 to 7 and in the most preferred embodiment the LogP value ranges from 2 to 7. In general, the LogP value of a compound can be determined experimentally, for example, by using reverse phase HPLC, see Yagam and Haraguchi, 2000, Chem. Pharm. Bulletin (Tokyo): 1973-7, or by using software models like the KowWin program, as described, for example, by Meylan and Howard. 1995, J. Pharm. Sci. 84: 83-92.

The present invention also comprehends the use of “amphiphilic” or “amphipathic” active agents, i.e., molecules with two distinct portions that differ in their affinity for solvents. One portion of the molecule has affinity for polar solvents, and is said to be hydrophilic and a second portion of the molecule has an affinity of non-polar solvents is said to be hydrophobic. The balance between the hydrophobic and hydrophilic portions in an amphipathic molecule (the “hydrophobic-lipophilic balance” or “HLB”) is employed to classify these molecules. The HLB values of commonly used amphipathic molecules are readily available from, for instance, the HANDBOOK OF PHARMACEUTICAL EXCIPIENTS (Pharmaceutical Press, 1994). The HLB of an amphiphilic active used in this invention can range from 1 to 15, more preferably from 5 to 13 and most preferably from 7 to 11.

An oleosome preparation containing the active may be obtained by simple mixing of the active ingredient with the oleosomes or by an optimized method that results in the selective partitioning of the active ingredient into oleosomes, as described by Murray et al. in PCT application 2005/030169.

Oleosome Preparations that Contain an Active Agent and a Release Control Agent

An oleosome preparation used in this context preferably comprises at least 5% oleosomes by volume. More preferably, the oleosome preparation comprises at least 10% or 20% or 30% or 40% or 50% or 60% or 75% oleosomes by volume.

To such an oleosome preparation, the release control agent preferably is added, in accordance with the invention, after partitioning of the active agent into the oleosomes. Upon its addition, the release control agent is dispersed in the oleosome preparation by simple mixing or stirring via, for example, an overhead stirrer of low shear (typically less than 500 rpm) or a magnetic stirrer and a stir bar. In a larger operation, a standard inline mixer or homogenizer can be used, as can any other means required to obtain a homogenous mixture, such as a pigment mill using a Cowles blade @ 3000 rpm for 15 to 20 minutes, provided that the mixing equipment is selected such that the shear forces generated during the mixing or stirring process are modest and the oleosomes remain intact.

As mentioned above, the amount of the aqueous phase within an oleosome preparation may vary. The release control agent preferably forms part of the aqueous phase of the oleosome preparation and its concentration preferably ranges between 1% and 99% by volume of the aqueous phase of the oleosome preparation. The concentration of release control agent is selected depending on the desired release kinetics and accordingly may be varied.

Pursuant to the invention, release control kinetics of the active may be adjusted to obtain a preparation with particularly desired release kinetics (“optimized”), by determining the release kinetics of the active agent at various concentrations of release control agent dispersed within a plurality of oleosome preparations. Accordingly, the present invention also encompasses an approach for obtaining an oleosome preparation comprising an active agent, where the release kinetics of the active agent have been optimized. This approach entails:
(i) preparing a plurality of oleosome preparations comprising an active agent and a release control agent, each oleosome preparation comprising a varying concentration of release control agent;
(ii) determining the release kinetics of the active agent in each oleosome preparation; and
(iii) selecting from the plurality of oleosome preparations an oleosome preparation with optimized active agent release kinetics.

To determine the release kinetics of the active in this context, the particle size of the oleosomes within an oleosome preparation may be determined using a particle size analyzer, such as the particle analyzer manufactured by Malvern Instruments Ltd. (Westborough, Mass.). The oleosome preparation can be applied to a surface area and, at different time points, samples may be analyzed for average or mean size of the particles and particle distribution patterns in the sample. In general, a change in average or mean size of the particles and/or in the distribution profile of the particles present within an oleosome preparation is indicative of degradation of the oleosomes and, hence, of release of the active agent from the oleosomes. With a volatile active agent, head space analysis may be employed, with a gas chromatographic device as an analytical tool, to assess the kinetics of release of active from an oleosome preparation. Alternatively, extraction using solvents such as hexane can selectively capture actives released from the oleosomes, allowing for quantification via spectrophotometric or other quantitative techniques.

Where glycerin is used as a control release agent, a 5% to 20% by volume glycerin concentration will result in a delay in release of the active agent from the oleosomes of 1 hour or more compared to a control preparation not comprising a release control agent, although this depends on the surface area, external conditions and the volumes applied.

In general, varying concentrations of the release control agent are expected to result in varying release kinetics relative to oleosome preparations not comprising a release control agent. For example, when increasing concentrations of PEG 200 are used, slower release kinetics are observed (see Example 6). In preferred embodiments the average rate of release of the active agent from the oleosomes is at least 15% less in the presence of the release control agent, when compared to the average rate of release of the active agent in the absence of the release control agent.

The present invention further encompasses novel oleosome preparations. In another of its aspects, therefore, the present invention further provides a composition comprised of (i) oleosomes, (ii) an active agent, and (iii) a release control agent. As noted, the release control agent is preferably a multihydric alcohol.

### Release of the Active Agent

An oleosome preparation of the invention may be applied to any surface area. Selection of a surface area in this regard is dependent primarily on the desired utility for a given finished formulation, comprising oleosomes (see further discussion, below). Upon application of the finished formulation to the surface area, the oleosome structure gradually weakens until it substantially disintegrates, thereby releasing the active agent. The kinetics of release of the active agent is a function of the disintegration rate of the oleosomes, which in turn is a function of the physical force used to apply the oleosomes to the surface area, as well as of the physical and chemical properties of the surface area and the conditions of exposure. In oleosome preparations comprising a multihydric alcohol release control agent, pursuant to the invention, oleosome stability generally is enhanced, resulting in a reduced rate of oleosome disintegration and, therefore, in active-agent release kinetics that are slower than those of an oleosome preparation lacking the release agent.

Release of the active agent may result from the application of physical force when the oleosome preparation is applied, for example, by rubbing of the oleosome preparation on skin or any other surface area or by drying of the oleosome preparation after it has been applied to the surface area. Where volatile active agents are used, such as fragrances, release of the active will result in evaporation of the active agents. Release of the active also may occur, after surface application of the oleosome preparation, due to the application of a physical force or a chemical compound to the surface area. In the latter instance, such a chemical would react with the oleosome resulting in disintegration of the oleosome structure and release of the active. In certain film-forming or coating applications, for example, release of the active may be initiated by abrasion of the film or coating or by contacting the coating or film with a chemical compound capable of initiating release of the active agent from the oleosomes.

Release kinetics can be characterized by the rate of release of the active agent or by the amount of the active agent released. The latter amount can be determined via analytical methods such as hexane extraction, which measures the amount of active agent outside the oil body at a given time point. The rate of release of an active agent can be determined through analytical measures such as particle size distribution changes over time, which reflects the distribution of particle size in the sample. Changes in the particle size of a sample indicate disintegration of oleosomes and, hence, the release of the active agent. Calculating the amount or rate of release over time provides the average rate of release.

In addition to the release kinetics of the active(s), certain other properties of an oleosome preparation may be altered in accordance with the invention. Illustrative of these properties are electrical conductivity, surface tension, and the ability to form a film or coating. These may be altered, as may be the rate of wicking or capillary action on surfaces such as fibers and hair.

### Utility of the Oleosome Preparation

The phrase “finished formulation” is used here to denote such a formulation, ready for its intended final use. An oleosome preparation obtained in accordance with the present invention may be used as an ingredient to prepare a multitude of finished formulations, as outlined, for example, in the Deckers Patents and in PCT applications 2005/030169 and 2005/097059, by the addition to the oleosome preparation of one or more additional compounds.

The formulations may take any of a wide array of forms, including but not limited to a cream, a gel, a lotion, a waxy solid, an ointment, a salve, a paste, a spray, or a milk. Advantageously, the formulation of such products is performed in the absence of exogenous emulsifiers. Finished formulations in accordance with the present invention are exemplified by formulations for topical application to the surface area of a mammal, such as personal care products, cosmetic products, topically applied pharmaceutical products, skin care products, cosmeceutical products, dermatological products, and topically applied veterinary products. Other products that may be formulated using the oleosome preparations obtained in accordance with the present invention are food, nutraceutical products, and nutritional supplements, as well as agriculture products, household products, inks, coatings, paints, pharmaceutical and industrial products.

### EXAMPLES

### Example 1

### Obtaining an Oleosome Preparation from Safflower

This example describes the recovery of the oleosome fraction from safflower. The resulting preparation contains intact washed oleosomes.

Seed decontamination. A total of 45 kg of dry safflower (Carthamus tinctorius) seed was washed twice using approximate 120 L of 65° C. tap water and once using approximately 120 L of about 15° C. tap water. The washing was carried out in a barrel with screen mesh to separate the waste water.

Grinding of seeds. The washed seeds were poured through the hopper of a colloid mill (Colloid Mill, MZ-130 (Fryma); capacity: 350 kg/hr), which was equipped with a MZ-130 crosswise toothed rotor/stator grinding set and top loading hopper, while approximately 100 L of 45 mM sodium bicarbonate buffer of pH 8.2 was supplied through an externally connected hose prior to milling. Operation of the mill was at a gap setting of 1R, chosen to achieve a particle size less than 100 micron at 18° C. and 30° C. All 45 kg of seeds were ground in 10 minutes

Homogenization and removal of solids. The resulting slurry was pumped into a knife in-line homogenizer (Dispax Reactor® DR 3-6/A, IKA® Works, Inc.) at a speed about 7 L/min. The output slurry was directly fed into a decantation centrifuge (NX-314B-31, Alfa-Laval) after bringing the centrifuge up to an operating speed of 3250 rpm. In 25 minutes approximately 160 kg of seed ground slurry was decanted. A Watson-Marlow (Model 704) peristaltic pump was used for slurry transfer in this step.

Oleosome separation. Separation of the oleosome fraction was achieved using a disc-stack centrifuge separator (SB 7, Westfalia) equipped with a three phase separating and self-cleaning bowl and removable ring dam series; maximum capacity: 83 L/min; ring dam: 69 mm. Operating speed was at ˜8520 rpm. A Watson-Marlow (Model 704) peristaltic pump was used to pump the decanted liquid phase (DL) into the centrifuge after bringing it up to operating speed. This results in separation of the decanted liquid phase into a heavy phase (HP1) comprising water and soluble seed proteins and a light phase (LP1) comprising oil bodies. The oleosome fraction, which was obtained after one pass through the centrifuge, is referred to as an unwashed oleosome preparation. This unwashed oleosome fraction was then passed through a static inline mixer, mixing with, 45 mM sodium bicarbonate buffer (pH 8.2, 35° C., 4 L/min) into a second disc-stack centrifuge separator (SA 7, Westfalia); maximum capacity: 83 L/min; ring dam: 73 mm. Operating speed was at ˜8520 rpm. The separated light phase (LP2) comprising oleosome was then passed through another static inline mixer mixing with 45 mM sodium bicarbonate buffer (pH 8.2, 35° C., 4 L/min) into the third disc-stack centrifuge separator (SA 7, Westfalia); maximum capacity: 83 L/min; ring dam: 75 mm. Operating speed was at ˜8520 rpm. The entire procedure was carried out at room temperature. The preparations obtained following the second separation are all referred to as the washed oleosome preparation.

### Example 2

### Preparing a Safflower Oleosome Preparation Comprising a Release Control Agent

To 100 g of a 75% solids dispersion of safflower oleosomes was added with low shear mixing, 25 g of pure glycerin (>99% purity). This reduced the total oleosome level to 60%. The pH of this dispersion is typically >8.5. Addition to the dispersion with low shear stirring of 1.25 g of a commercial preservative Geogard Ultra which consists of a blend of glucono delta lactone and benzoic acid reduces the pH of the dispersion to 4.2-4.5. The dispersion passed microbial challenge testing and no leakage of the oleosomes was observed even after 6 months at 25° C.

### Example 3

### Comparison of Physical Stability of a Safflower Oleosome Preparation with and Without Release Control Agent

A total of 100 μl of a safflower oleosome preparation with and without glycerin as a release agent was spread over the inside surface of a 50 ml centrifuge tube lid. The samples were allowed to air dry for varying amounts of time at room temperature. Samples then were resuspended in 20 ml of 0.025M bicarbonate buffer, and a 1 ml aliquot was analyzed by laser diffraction to assess particle size. Standard oil bodies are the original formulation with bicarbonate buffer and preserved with Neolone and Glycacil. The glycerin oil bodies are formulated with glycerin and Geogard Ultra.

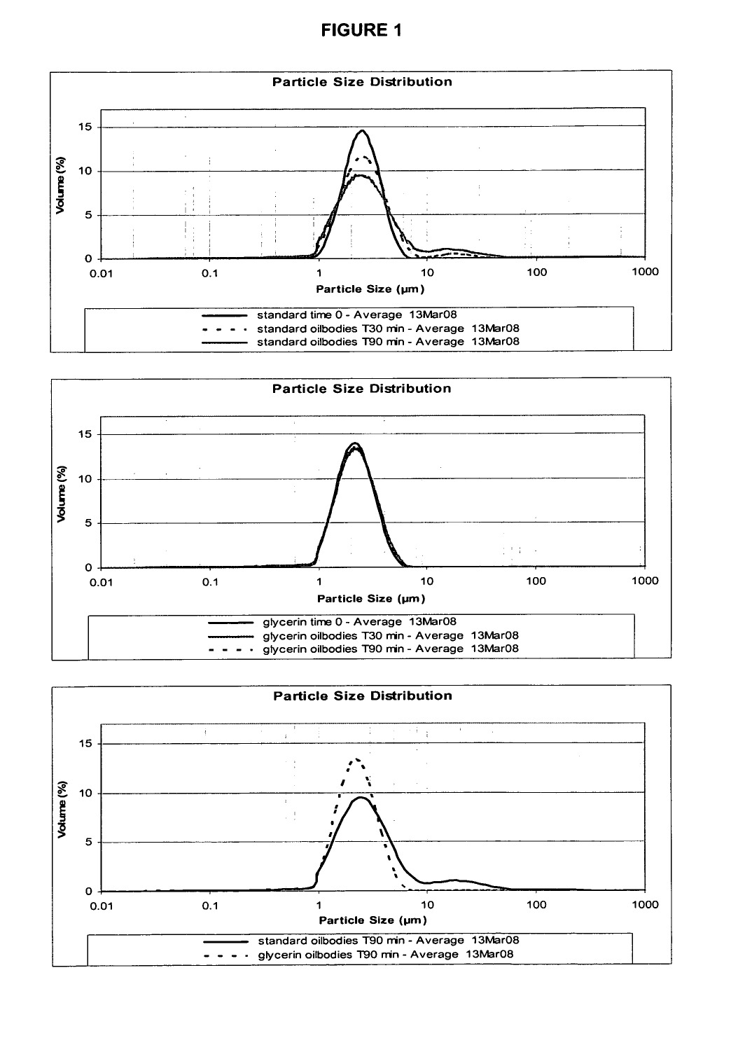

As shown in FIG. 1, in preparations comprising oleosomes and glycerin as a release control agent, the particle size distribution remains unchanged during the 90 minute drying period. In contrast, in preparations comprising oleosomes without glycerin, during the 90 minute drying period the particle size gradually changes and larger size particles appear at the expense of the smaller size particles, indicative of a disintegration of the oleosome structure and release of the lipophilic contents of the oleosome.

### Example 4

### Comparison of Kinetics of Release of OMC from an Oleosome Preparation with and without a Release Control Agent

To a 90 g sample of a 75% dispersion of oleosomes, enough OMC (2-Ethylhexyl trans-4-methoxy-cinnamate, Sigma Aldrich) was added to yield a final mixture that was 15% dry weight or 10% sample weight OMC. The mixture then was stirred, using a low speed mixer for 2 minutes at room temperature, to facilitate absorption of the OMC into the oleosomes.

The sample was dispensed into two 5 g samples. The first sample was diluted with 0.5 g of distilled water and mixed for 5 minutes. The final ratio of loaded oleosomes to diluent is 68/32.

The second sample was diluted with 0.5 g of pure glycerin and mixed for 5 minutes, so as to yield a final mixture in which the ratio of oleosomes/glycerin/water is 68%/9%/23%, respectively.

Aliquots (90-92 mg) were removed from each of the two samples and spread over a 1.2 cm² area on tin foil. The tin foil pieces containing the samples were exposed to 35° C. in an incubator for 1, 2, 3 or 4 hours. The tin foil was placed in a glass extraction tube and extracted with 10 ml of pure hexane until the oleosomes were visually resuspended. The samples were centrifuged and the extract removed to a fresh tube.

OMC can be detected by spectrophotometry using a wavelength of 310 nm. Several dilutions of the extract were required for spectrophotometric assessment. The absorbance values obtained were then corrected to account for the dilutions. The results of the hexane extracts are given in Table 1. Tracking the concentration of OMC released over time using hexane extractions (see below) showed that the oleosome sample, formulated with glycerin as a release control agent, retained the OMC for a significantly longer time than the sample diluted with water.

TABLE 1
Relative corrected absorbance values for
hexane-extracted, OMC-loaded oleosomes

| Release Agent | 0 hr | 1 hr | 2 hr | 3 hr | 4 hr |
|---|---|---|---|---|---|
| 10% Glycerin | 4.9 | 11.0 | 7.5 | 7.7 | 9.7 |
| 10% Water (control) | 6.7 | 91.6 | 57.9 | 55.1 | 58.5 |

Oleosomes were exposed to release conditions from 0 to 4 hours at 35° C. and then were extracted with hexane. The OMC content of the hexane extract was assessed spectrophotometrically at 310 nm. Absorbance values were adjusted to reflect the dilutions of the samples.

### Example 5

### Comparison of Glycerin and PEG 200 on the Kinetics of Release of OMC from an Oleosome Preparation

A sample containing 15% dry weight of OMC was prepared in accordance with the method described in Example 4. The loaded oleosomes were then mixed with 15% by weight glycerin or PEG 200 (polyethylene glycol 180-210 MW, Sigma Aldrich). Samples of each oleosome preparation (90-92 mg) were evenly distributed over 1.2 cm² of tin foil and incubated at 35° C. for 2 hours.

Oleosome degradation or release of OMC was assessed using laser diffraction particle size analysis. The tin foil containing the sample was slightly folded with the sample inward and placed in a 15 ml conical tube containing 10 ml of 25 mM sodium bicarbonate buffer. The sample was shaken until the oleosomes were determined by visual analysis to be removed from the tin foil. The sample was then analyzed for particle size distribution using the Mastersizer 2000 (Malvern Instruments) particle size analyzer.

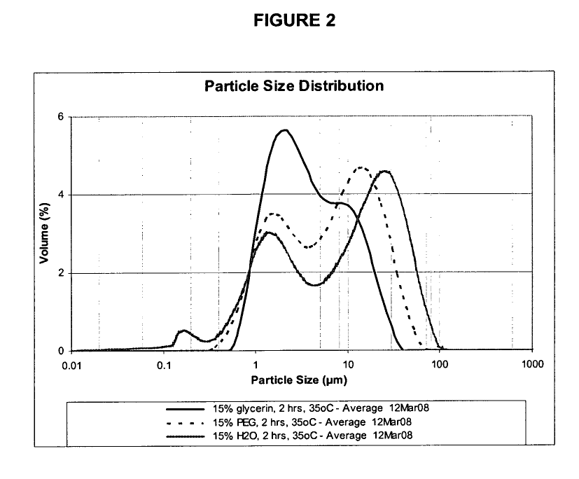

The results are summarized in Table 2 and FIG. 2. The parameter “d (0.5)” denotes the size (μm) below which 50% of the particle distribution falls. The results demonstrate that the type of control release agent used affects the kinetics of release of an active agent from an oleosome preparation. Glycerin, a heavier weight molecule (specific gravity of ˜1.26, compared to ˜1.13 for PEG 200), better protects the loaded oleosomes from degradation, resulting in slower release of the OMC from the oleosome than PEG.

TABLE 2
Average particle size of OMC-loaded oleosomes
mixed with 15% PEG 200 or glycerin

| Sample Name | d (0.5) |
|---|---|
| 15% Glycerin 2 hrs 35° C. | 3.674 |
| 15% PEG 2 hrs 35° C. | 7.283 |
| 15% Water 2 hrs 35° C. | 10.93 |
| 15% OMC loaded oleosomes | 2.868 |
| Unloaded Hydresia 2008-094 | 2.646 |

The “2008-094” sample was constituted of the unloaded oleosomes, and the 15% OMC was the starting stock of loaded oleosomes before addition of release control agent. Both controls were not exposed to release conditions.

### Example 6

### Comparison of Kinetics of Release of OMC from an Oleosome Preparation with Varying Concentrations of PEG 200 as a Release Control Agent

A sample containing 15% dry weight of OMC was prepared in accordance with the method described in Example 4. The loaded oleosomes were then mixed with 5%, 10%, 15% or 20% by weight PEG 200 (polyethylene glycol 180-210 MW, Sigma Aldrich) or distilled water (control). Samples of each oleosome preparation (90-92 mg) were evenly distributed over 1.2 cm² of tin foil and incubated at 35° C. for 2 hours.

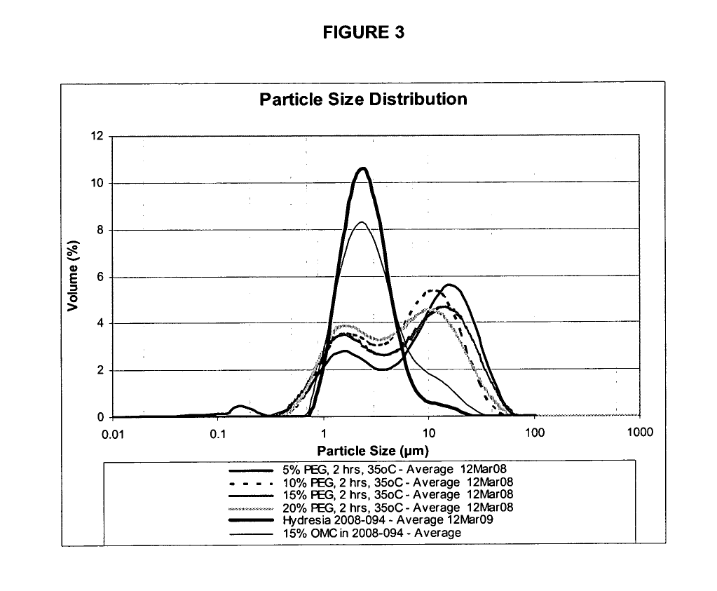

Oleosome degradation was assessed as described above, using laser diffraction particle size analysis. The results are summarized in Table 3 and FIG. 3. As before, “d (0.5)” is the size in μm below which 50% of the particle distribution falls.

The results demonstrate that the employed concentration of control release agent affects the kinetics of release of OMC from an oleosome preparation. Higher concentrations of the release control agent (PEG) resulted in lower d (0.5) values, indicating there was less deterioration of the oleosomes and, hence, a slower rate of release of OMC. The sample with the lowest concentration (5%) of release agent had the largest d (0.5) value, indicating the most damage to the oleosomes.

TABLE 3
Average particle size of OMC-loaded oleosomes mixed with
5, 10, 15 or 20% PEG 200 following 2 hours at 35° C.

| Sample Name | d (0.5) |
|---|---|
| 5% PEG 2 hrs 35° C. | 9.894 |
| 10% PEG 2 hrs 35° C. | 6.741 |
| 15% PEG 2 hrs 35° C. | 7.283 |
| 20% PEG 2 hrs 35° C. | 5.699 |
| 15% OMC loaded oleosomes | 2.868 |
| Unloaded Hydresia 2008-094 | 2.646 |

### Example 7

### Rate of Release of OMC from an Oleosome Preparation with Glycerin or PEG 200 as a Release Control Agent

A sample containing 15% dry weight of OMC was prepared in accordance with the method, described in Example 4. The loaded oleosomes then were mixed with 10% by weight of either glycerin or PEG 200 (polyethylene glycol 180-210 MW, Sigma Aldrich). Samples of the oleosome preparation (90-92 mg) were evenly distributed over 1.2 cm² of tin foil and incubated at 35° C. for 1, 2, 3 or 4 hours.

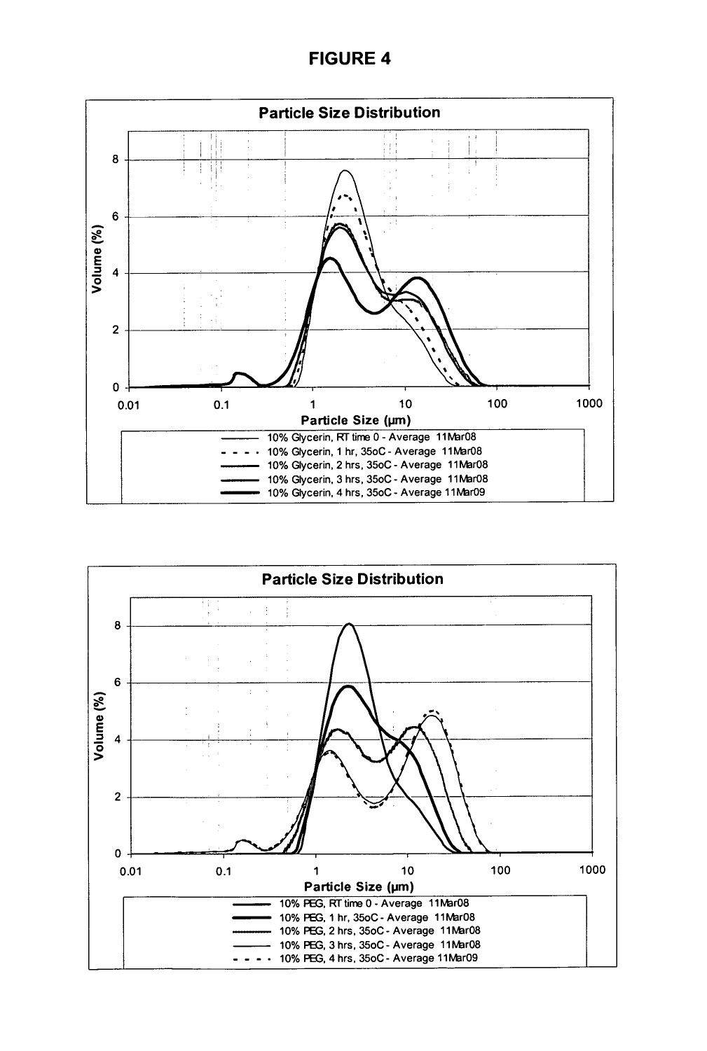

Oleosome degradation was assessed as before. The results, summarized in Table 4, Table 5 and FIG. 4, demonstrate that, in the presence of PEG 200, the average rate of release of OMC from the oleosome preparation was approximately 18% less than the rate of release from the oleosome preparation without a release control agent. In the presence of glycerin, the average rate of release of OMC from the oleosome preparation was approximately 93% less than the rate of release from the oleosome preparation without a release control agent (see Table 4). Longer incubation time resulted in higher d (0.5) values and a shift to the right of the particle size distribution curve (see Table 5 and FIG. 4), indicating increased deterioration of the oleosomes in the preparation over time.

TABLE 4
Comparison of the amount of OMC released from loaded
oleosomes with a release control agent (glycerin or PEG 200)
to oleosomes without a release control agent (water control)

| | | | | | | | | | % |
|---|---|---|---|---|---|---|---|---|---|
| | | | | | | | | | Difference |
| | | | | | | | | Avg | in Rate |
| | | | | | | Avg. | % | Rate of | (compared |
| Release Agent | 0 hr | 1 hr | 2 hr | 3 hr | 4 hr | Release | Release | Release | to Control) |
| 10% glycerin | 4.9 | 11.0 | 7.5 | 7.7 | 9.7 | 8.98 | 13.6 | 1.0 | 93.2 |
| 10% PEG | 7.1 | 55.8 | 53.8 | 47.9 | 64.8 | 55.6 | 84.5 | 12.1 | 18.2 |
| 10% water (control) | 6.7 | 91.6 | 57.9 | 55.1 | 58.5 | 65.8 | 100 | 14.8 | 0.0 |

Oleosomes were exposed to release conditions of 35° C. from 0 to 4 hours, and then were extracted with hexane. The OMC content of the hexane extract was assessed spectrophotometrically at 310 nm. Absorbance results were adjusted to reflect the dilutions of the samples. The parameter “% release” was calculated from comparison to the OMC released in the water control, which is considered to be 100%. “Average rate of release” is the average amount of active released over time.

TABLE 5
Average particle size of OMC-loaded oleosomes mixed
with 10% PEG following up to 4 hours at 35° C.

| | | % Change in |
|---|---|---|
| | | Particle Size |
| Sample Name | d (0.5) | Distribution |
| 10% PEG 35° C. time 0 | 2.91 | 100 |
| 10% PEG 35° C. 1 hour | 3.68 | 126 |
| 10% PEG 35° C. 2 hours | 5.18 | 178 |
| 10% PEG 35° C. 3 hours | 8.19 | 281 |
| 10% PEG 35° C. 4 hours | 8.74 | 300 |

While the present invention has been described with reference to what are presently considered to be preferred examples, it is to be understood that the invention is not limited to the disclosed examples. To the contrary, the invention is intended to cover various modifications and equivalent arrangements included within the spirit and scope of the appended claims.

All publications, patents and patent applications are incorporated by reference in their entirety to the same extent as if each individual publication, patent, or patent application was specifically and individually indicated to be incorporated by reference in its entirety.

1. A method for the release of an active agent from an oleosome, comprising:
(i) providing an oleosome preparation comprising an encapsulated active agent and a release control agent; and
(ii) applying the oleosome formulation to a surface such that the active agent is released from the oleosome formulation.

2. The method according to claim 1, wherein the average rate of release of the active agent from the oleosomes is at least 15% less in the presence of the release control agent, when compared to the average rate of release of the active agent in the absence of the release control agent.

3. The method according to claim 1, wherein the release control agent is a multihydric alcohol.

4. The method according to claim 3, wherein the multihydric alcohol is a non-aromatic diol, a non-aromatic triol, a non-aromatic polyol or a non-halogenated multi-hydric alcohol.

5. The method according to claim 1, wherein the release control agent is glycerin.

6. The method according to claim 1, wherein the release control agent is PEG.

7. The method according to claim 5, wherein the glycerin or PEG is present in the formulation in an amount of 5% to 20% by volume.

8. The method according to claim 1, wherein the surface area is skin.

9. A controlled release oleosome formulation comprising an active agent encapsulated in oleosomes and a release control agent.

10. The formulation according to claim 9, wherein the average rate of release of the active agent from the oleosomes is at least 15% less in the presence of the release control agent, when compared to the average rate of release of the active agent in the absence of the release control agent.

11. The formulation according to claim 9, wherein the release control agent is a multihydric alcohol.

12. The formulation according to claim 11, wherein the multihydric alcohol is a non-aromatic diol, a non-aromatic triol, a non-aromatic polyol or a non-halogenated multi-hydric alcohol.

13. The formulation according to claim 9, wherein the control release agent is glycerin.

14. The formulation according to claim 9, wherein the control release agent is PEG.

15. The formulation according to claim 13, wherein the glycerin or PEG is present in the formulation in an amount of 5% to 20% by volume.

16. A finished formulation comprising a composition according to claim 9.

17. The finished formulation according to claim 16, wherein said finished formulation is a personal care product, a cosmetic product, a topically applied pharmaceutical product, a skin care product, a cosmeceutical product, a dermatological product, a topically applied veterinary product, a food product or an industrial product.

18. A method for obtaining an oleosome preparation comprising an active agent, the release kinetics of which have been optimized, the method comprising:
(i) preparing a plurality of oleosome preparations, each comprising an active agent and a release control agent wherein each oleosome preparation comprises a different concentration of release control agent;
(ii) determining the release kinetics of the active agent in each oleosome preparation; and
(iii) selecting from the plurality an oleosome preparation with optimized active agent release kinetics.
